(19) United States
(12) Patent Application Publication
(10) Pub. No.: US 2005/0277973 A1
(43) Pub. Date: Dec. 15, 2005

(54) Ultrasonic pigment application device

(76) Inventors: Joseph Huang, Walnut, CA (US); John Dreher, Mira Loma, CA (US)

Correspondence Address: BUHLER & ASSOCIATES;SUITE 100, 1101 CALIFORNIA AVENUE, CORONA, CA, 92881, US

(21) Appl. No.: 10/857,729

(22) Filed: Jun. 1, 2004

(51) Int. Cl. A61B 17/34

(52) U.S. Cl. 606/185

(57) ABSTRACT

An ultrasonic skin pigment application device where the device moves the needle applying the pigment at an ultrasonic rate. The device can be a self contained, self-powered, or externally powered. The invention may also include replaceable needles, and or a pigment reservoir. The device may also have adjustments for stroke length, stroke force, and stroke rate. The needle moving mechanism may be either an ultrasonic linear piezo drive or solenoid type drive mechanism or actuator.

### FIELD OF THE INVENTION

The present invention relates generally to a pigment application device. More specifically the invention relates to an ultrasonic tattoo application invention intended for the application of pigment or coloring to within the skin, intradermal or other surface.

### BACKGROUND OF THE INVENTION

Many people want their skin marked with color that is imbedded into their skin or intradermas. For some people the skin marking is a tattoo and is performed to add a picture or pattern onto the skin. For other people the marking of the skin is to apply permanent or semi-permanent coloring to eliminate the need to apply make-up. This type of coloring can be applied to the eyelids, eyebrows, lips, cheeks or other areas that a person would apply color. Most tattoo or skin marking equipment is performed by applying color to the surface of the skin or a needle, and pressing the needle through the surface of the skin, to push the color into the intradermas. Current tattoo devices have the needle attached to a motor that drives the needle in and out of the surface of the epidermis. With each stroke of the needle pushes some coloring from the surface of the epidermis or from a pigment reservoir below the surface of the epidermis where the color in retained. Prior inventions have used a needle that is depressed by the operators hand to push the coloring into the skin. Other prior inventions use a rotating mechanism to translate the needle into and out of the skin. What is needed is a pigment application device that operates at an ultrasonic rate so the pigment can be applied at higher rate.

Pat. No. 4,031,783 issued to Paul et al. discloses a remote driven tattoo etching machine where the needle is driven with a rotating mechanical motor that is external to the hand held tattooing device. While this invention provides a mechanism for tattooing the surface of the skin, the device relies upon a mechanical rotating motor to oscillate the marking needle.

Pat. No. 4,665,912 issued to Burton discloses a manually oscillated skin marking device there the operator depresses a spring returned button on the back of a pen shaped skin marking device. When the button is depressed, a needle pushes dye into the skin. While this invention provides a mechanism for tattooing the surface of the skin, the device relies upon the operator to oscillate the marking needle.

Pat. No. 4,796,624 issued to Trott et al. discloses a motor driven lash liner where an ink reservoir is in contact with the needle. A rotating mechanical motor oscillates the needle within the reservoir into and out of the surface of the skin. While this invention provides a mechanism for tattooing the surface of the skin, the device relies upon a mechanical rotating motor to oscillate the marking needle.

Pat. No. 5,776,158 issued to Chou, discloses a pigment applicator with a detachable needle. The needle is operated with a rotating mechanical motor to oscillate the marking needle. While this invention provides a mechanism for tattooing the surface of the skin, the device relies upon a mechanical rotating motor to oscillate the marking needle.

While all these prior inventions can perform the function of marking skin, none of them utilizes oscillating the needle with an ultrasonic actuator. The ultrasonic actuator allows both a faster application rate of the coloring and, less vibration of the marking devise that allows better control and detail for skin marking.

### BRIEF SUMMARY OF THE INVENTION

It is an objective of the present invention to provide a device for pigmenting (tattooing) the skin where the device moves the pigment-applying needle at an ultrasonic rate. The application of pigment to the skin can be a painful experience. The experience is painful because a needle pushes pigment into the surface of the skin. The longer the application takes the longer a person is subjected to the needle piercing the skin. If the area is large, the time required to apply the pigment can last several hours.

The ultrasonic rate of moving the needle provides a superior application of the pigment for a number of reasons. The needle is moved at a faster rate so the application of pigment is applied at a higher rate. Because the application of the pigment is faster, the time to apply the pigment to the skin is reduced. Operating the invention in the ultrasonic range provides a less vibration of the device as the operator applies pigment. Since the vibration is reduced, the operator has better control of the device.

Unlike mechanical motors, ultrasonic motors or actuators can be operated in a number of different frequencies forces and stroke lengths. Adjustments can be located on the device that allow the operator to adjust the speed, frequency, or number of strokes per second of the device to allow the operator to have greater control over the pigment application process. The stroke force can also be adjusted so the device rate can limit how hard the needle is pushed into the skin. The stroke length may also be adjustable to control how deep the pigment is pushed into the skin. For thick callused skin, the operator may want to push the pigment deep. For soft facial skin, the operator may want to push the pigment just under the surface of the skin. The depth adjustment may also allow also allow a customer to determine how long the pigment may be present until the epidermis naturally sheds the pigmented layer(s) of skin.

The need for an ultrasonic tattooing device that has the features listed has long been present in the pigment marking industry. The proposed invention satisfies these needs. Various objects, features, aspects, and advantages of the present invention will become more apparent from the following detailed description of preferred embodiments of the invention, along with the accompanying drawings in which like numerals represent like components.

### BRIEF DESCRIPTION OF THE DRAWINGS

FIG. 1 is an isometric view of the invention showing one embodiment of the components.

FIG. 2 is a detailed view of the drive mechanism using a piezo drive mechanism.

FIG. 3 is a detailed view using a ferrous core and an inductive drive mechanism.

FIG. 4 is a detail of a quad needle

FIG. 5 is a block diagram of the electrical drive mechanism.

FIG. 6 is a schematic circuit diagram of the electrical drive mechanism.

### DETAILED DESCRIPTION OF THE INVENTION

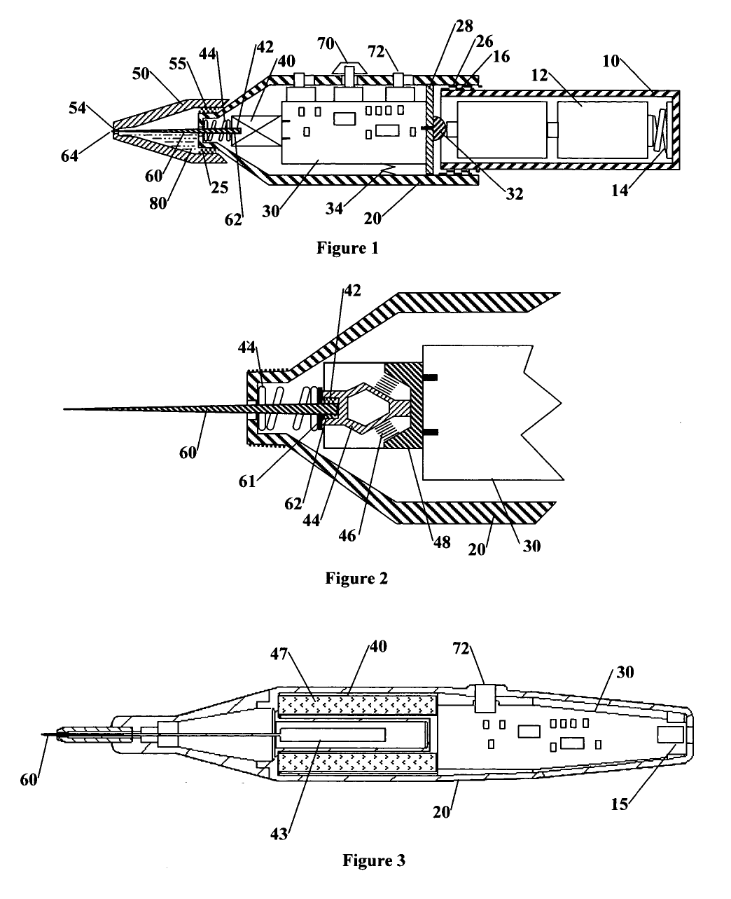

Referring first to FIG. 1, that shows an isometric view of the invention showing one embodiment of the components. This figure shows a general view of the components that will be described in more detail with the additional figures. FIG. 1 shows the invention in a self-contained version where all the components are enclosed in a single handheld unit. Other embodiments are contemplated that include an external power supply and or control electronics. A single two or three conductor wire connects from the external power supply and control electronics to the handheld ultrasonic pigment marking device. The figure includes a rear enclosure member 10 that contains a power source. The power source in this embodiment is with two “C” batteries item 12. The batteries are pushed into the positive contact terminal 32 with spring 14 located in the back of the rear enclosure member. The rear enclosure member is threaded into the body of the main body 20 of the pigment application device with threads from the rear enclosure member 16 and threads located in the main body 26. An insulator 28 separates the rear-threaded section of the main body from the control electronics.

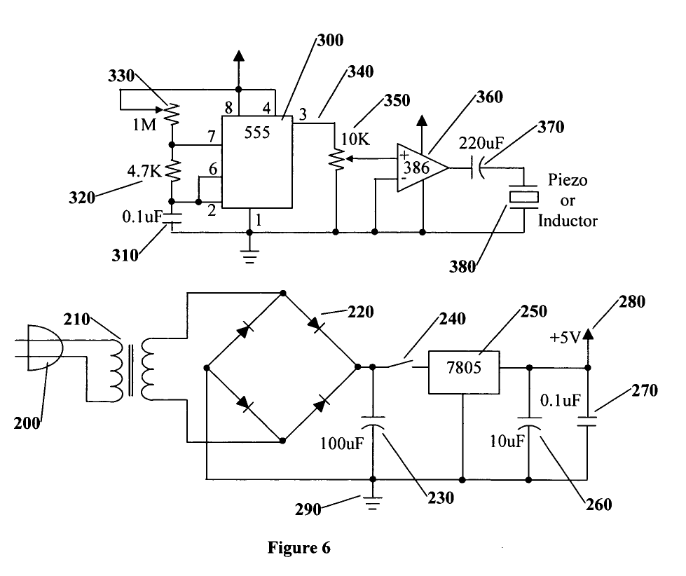

In the preferred embodiment, the control electronics 30 are contained in the main body. One possible schematic for the control electronics is shown in FIG. 6. The control electronics are shown enclosed within the main body, but the control electronics may be located external to the pigment application device. Locating the control electronics will reduce the weight of the pigment device. Another embodiment may include a foot-activated control or switch so the operator can keep both hands free to use the pigment application device. Connection to the ground of the power supply is made by item 34 that provides both a grounding connection and keeps the electronics biased to one side of the housing. The electronics can have multiple adjustments for needle force, stroke and rate. These adjustments can be performed using switch 72 and or an adjustable knob or dial 70. The functions and controls will be explained in more detail in FIG. 6.

The electronics are connected to an ultrasonic linear drive mechanism or actuator 40. Two types of ultrasonic linear drive actuators are shown in FIGS. 2 and FIG. 3. The side of the main body opposite the batteries has a tapered section that is threaded 25 into a mating threaded section 55 of a replaceable front enclosure 50. The back end 62 of the needle 60 is inserted into the ultrasonic linear drive at item 42. The needle is held against the ultrasonic linear drive actuator with spring 44. The spring keeps the needle retracted against the ultrasonic linear drive. The front enclosure member 50 is shown as a replaceable housing, but the housing may be a permanent part of the housing. At the end of the front enclosure member a small opening 54, that guides the tip of the needle 64. Pigment may be added into the front enclosure 80. As the needle retracts into the front enclosure, a small amount of pigment is added to the needle. As the needle moves out of the front enclosure, the pigment is drawn from the front enclosure and pushed from the needle under the surface of the skin.

To eliminate the trouble of cleaning and sterilizing the needle, the needle and front enclosure can be removed and discarded. A new needle can then be inserted, pigment added to a new front enclosure, and the front enclosure screwed onto the main body. This allows a fast and sterile change of pigment.

Referring now to FIG. 2, that provides a detailed drawing of one embodiment of the needle drive mechanism. In this embodiment, the drive mechanism is a linear piezo drive mechanism. The linear piezo drive 40 is shown inside the main body 20. The control electronics are shown connected to the linear piezo drive. A number of companies manufacture linear piezo drive motors such as National Instruments and Physik Instruments. An embodiment of the linear piezo drive is shown where the piezo elements 46 push from back of the piezo drive 49 against a pyramid shaped structure 48. As the piezo elements expand, they push in on the sides of the pyramid structure, the motion is amplified and the needle is pushed forward. The needle 60 is threaded 62 into the linear piezo drive 42. The needle is held against the ultrasonic linear drive with spring 44. The spring keeps the needle retracted against the ultrasonic linear drive. A washer 61 provides a flat surface for the spring to keep the end of the linear piezo drive in the retracted position and the needle retracted within the front enclosure.

Referring now to FIG. 3, that provides a detailed drawing of an alternative linear needle drive mechanism 40. In this embodiment, the drive mechanism is a ferrous core 43 and an inductive drive mechanism 47. The linear drive 40 is shown inside the main body 20. The control electronics 30 are shown connected to the core and inductive drive. A number of companies manufacture linear solenoid drives of this type. The core is a thin material to reduce mass or inertia that is being moved. The inductive drive consists of multiple turns of wire. The inductive drive energizes and creates a magnetic field that pushes the core out of the inductive field. The needle 60 is connected to the ferrous core 43. In this figure, an external power supply is connected to the control electronics at jack 15. Switch 72 turns the invention on and off. In this embodiment the needle is moved at a fixed rate, stroke.

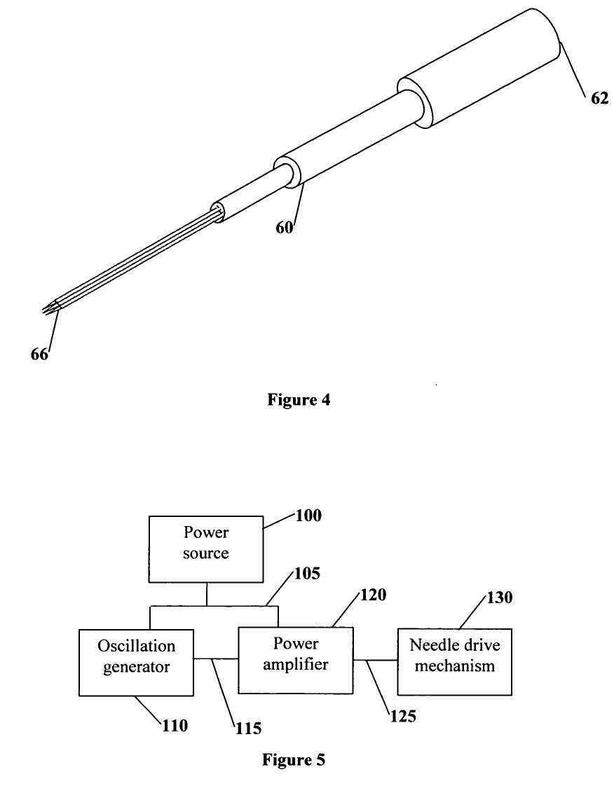

Referring now to FIG. 4 that shows a quad or four-tip needle 60. The needle has a rear section 62 that connects is retained or screws into the linear drive mechanism. The main body of narrows in steps to the quad needle assembly as shown in this figure, but the body may taper to the quad needle assembly. The quad needle 66 shows a collection of four needle tips, but the needle may be as few as one point to as many as four or more points that allow application of the pigment at a higher rate.

Referring now to FIG. 5 that show a block diagram of the electrical drive mechanism. This block diagram can be used for either a hardwired logic as shown and described in FIG. 5, or as a microprocessor controller drive logic where the microprocessor controls the rate, force, position, and or stroke of the needle. The power source 100 provides power to both the oscillator 110 and the power amplifier 120 with connection 105. In the preferred embodiment the power source is from batteries. The oscillator provides a square, sine, saw, triangular or other wave shape. The frequency of the oscillation may be an adjustable or fixed frequency. The oscillation generator many be provided by a micro-controller or microprocessor that provides a digital or analog output for the frequency of oscillation. The oscillation generator is connected to the power amplifier 120 with connection 115. The power supply can output a fixed or variable amount of power with connection 125 to the needle drive mechanism 130. The needle drive mechanism can be a variety of types that output a linear motion. In the preferred embodiment, the output is a piezo linear drive mechanism that moves the needle out and back. Strain and or position gauges may be placed on the needle drive mechanism to provide feedback regarding the position and or force of the needle.

Referring now to FIG. 6 that show a schematic circuit diagram of the electrical drive mechanism. The schematic consists of a user interface portion items 240, 330 and 350, frequency generator 300, and a linear drive 380. The analog portion can alternately be performed with a microprocessor programmed to read adjustable inputs from the user interface, and make the required outputs and commands to the drive portion of the circuit. In this embodiment, the schematic shows a wall plug 200 connected to transformer 210 but the power source could be batteries contained within the pigment application device. The transformer is a step down transformer that provides a lower voltage. The output of the transformer goes into diodes 220 of a full wave bridge. The full wave bridge provides converts the sine wave output from the transformer into a ground and positive voltage. The voltage is filtered with capacitor 230. Switch 240 allows the power to the circuit to be turned on and off. The voltage then is regulated with the voltage regulator 250 to a fixed voltage. The regulator in this embodiment is a five-volt regulator. The output from the regulator is filtered using filtering capacitors 260 and 270.

The frequency generator 300 is connected to the regulated voltage 280 and ground connection 290. The frequency generator includes a number of components resistor 320 and capacitor 310 that allow the range of adjustment for the frequency to be set. Variable resistor 330 allows the adjustment of frequency. The selected components allow for an output frequency from about one thousand cycles per second to about 20 thousand cycles per second. The frequency is available at item 340. This output is then sent into variable resistor 350 that sets the amount of signal that will be amplified. This adjustment sets the amount of force, power or energy that will be provided to the linear drive 380. The signal from the variable resistor 350 goes into the power amplifier 360 that provides the power for the linear drive that moves the needle. The output from the power amplifier is filtered with capacitor 370 and then connected to the linear drive 380. In the schematic, the linear drive is a piezo linear drive, but any type of linear drive system will work that provides linear motion from the output of the power amplifier.

Thus, specific embodiments and applications for an ultrasonic pigment application device have been disclosed. It should be apparent, however, to those skilled in the art that many more modifications besides those described are possible without departing from the inventive concepts herein. The inventive subject matter, therefore, is not to be restricted except in the spirit of the appended claims.

1. An ultrasonic skin pigment application device for applying pigment into the skin comprising:
a power source connected to an ultrasonic drive actuator; and
at needle connected to the drive actuator for the purpose of applying pigment into the skin.

2. The ultrasonic drive actuator from claim 1 wherein the actuator is an ultrasonic linear drive.

3. The ultrasonic drive actuator from claim 1 wherein the actuator is a ferrous core inside an inductive drive.

4. The drive actuator from claim 1 wherein the stroke of the needle is adjustable.

5. The drive actuator from claim 1 wherein the rate of the movement of the needle is adjustable.

6. The drive actuator from claim 1 wherein the force of the actuation of the needle is adjustable.

7. The pigment application device from claim 1 further includes a reservoir of pigment.

8. The power source from claim 1 further includes control circuitry to adjust the power and or stroke rate of the needle.

9. The skin pigment applicator from claim 1 wherein the applicator further includes a power supply.

10. The skin pigment applicator from claim 1 wherein the applicator includes a power supply and control apparatus that is attached to the drive apparatus with a flexible electrical cord.

11. The needle from claim 1 wherein the needle is replaceable.

12. The pigment application device from claim 1 that further includes a replaceable front housing.

13. The control circuitry from claim 8 that further includes sensors for force and or position.

14. The control circuitry from claim 8 that further includes a user interface display

15. The display from claim 14 wherein the display provides numerical of graphical information.

16. A method of applying pigment within the skin of a person comprising:
applying pigment to the needle of an ultrasonic oscillating pigment application device;
contacting the epidermis of a person with the needle; and
oscillating the needle such that the pigment is pushed into the intradermas of the person.

17. The method of claim 16 wherein the ultrasonic pigment application device is oscillated with a ultrasonic linear drive.

18. The method from claim 16 wherein the needle and or the pigment is replaceable.

19. A needle for a pigment application device comprising:
a shaft connected to a pigment application device, and
more than one point is attached to one end of the shaft.

20. The more than one point from claim 19 wherein the needle comprises four points.
